(19) United States
(12) Patent Application Publication
(10) Pub. No.: US 2018/0044628 A1
(43) Pub. Date: Feb. 15, 2018

(54) Method for Biofilm Control and Treatment

(71) Applicant: The Regents of the University of California, Oakland, CA (US)

(72) Inventors: Gürol Suel, San Diego, CA (US); Jintao Liu, Irvine, CA (US)

(21) Appl. No.: 15/556,134

(22) PCT Filed: Mar. 11, 2016
(86) PCT No.: PCT/US16/21956

§ 371 Date: Sep. 6, 2017

Related U.S. Application Data
(60) Provisional application No. 62/132,561, filed on Mar. 13, 2015.

(51) Int. Cl. C12N 1/20; A01N 59/00

(52) U.S. Cl. CPC C12N 1/20; A01N 59/00; C12N 2500/35; C12N 2501/84; C12N 2500/05

(57) ABSTRACT

The invention provides a novel method for controlling and/or treating the growth of biofilms by promoting growth of the periphery cells and/or increasing nutrient consumption by the periphery cells so that the growth of peripheral cells is not only independent from nutrients (e.g., ammonium) provided by the interior cells of the biofilms, but also causes interior cells starved to death. The accessible peripheral cells are then treated and/or eliminated by biofilm control substance of antibiotics or toxic chemicals. Therefore, the invention method controls and eliminates both peripheral and interior cells within biofilms. The invention method can be used in various industries, such as clinical and dental medicine and medical equipment, food and oil industry, and water supply systems.

### CROSS-REFERENCE TO RELATED APPLICATIONS

This application claims priority to a U.S. Provisional Patent Application No. 62/132,561, filed Mar. 13, 2015, the entire contents and substance of which are hereby incorporated by reference as if fully set forth below.

### STATEMENT AS TO FEDERALLY SPONSORED RESEARCH

This invention was made with a government support under Grant No. GM088428 awarded by the National Institute of Health, and under Grant No. MCB-1450867 awarded by the National Science Foundation. The government has certain rights in this invention.

### FIELD OF THE INVENTION

The invention is directed to methods and materials that control and/or treat biofilms.

### BACKGROUND OF THE INVENTION

Cooperation and competition are complex social interactions that can play critical roles in biological communities. Cooperative behavior often increases the overall fitness of the population through processes such as division of labor and production of common goods¹⁻⁴. At the same time, individuals in a community compete with each other for limited resources, such as nutrients⁵⁻⁶. Cells that reside within a community can cooperate and also compete with each other for resources. It remains unclear how these opposing interactions are resolved at the population level.

Biofilms typically form under environmental stress conditions, such as nutrient limitation¹¹⁻¹³. As these bacterial communities grow larger, the supply of nutrients to interior cells becomes limited due to an increase in nutrient consumption associated with the growth of multiple layers of cells in the biofilm periphery. Severe nutrient limitation for interior cells is detrimental to the colony, since the sheltered interior cells are critical to the survival of the biofilm community in the event of an external challenge. This defines a fundamental conflict between the opposing demands for biofilm growth and maintaining the viability of protected (interior) cells (FIG. 1a). The identification of possible mechanisms that ensure the viability of the protected interior cells is fundamental to understanding biofilm development^{14, 15}.

Treating or controlling biofilm growth can be useful in a variety of applications including, but not limited to, clinical medicine, dental medicine, the food industry, the oil pipeline industry, water supply systems, and others. Many current strategies to control or treat biofilm formation (or attempts to eliminate biofilms) rely on exposing biofilms to toxic chemicals, such as hydrogen peroxide, or antibiotics. However, these approaches have had limited success, because cells deep within the biofilm interior are highly protected against such attacks by the many layers of peripheral cells. Accordingly, improved methods and materials are desired to control and/or treat biofilms.

### SUMMARY OF THE INVENTION

The invention provides a novel method of controlling and/or treating biofilms. In certain embodiments, the invention discovers an internal conflict within a microbial biofilm community: cells in the biofilm periphery not only protect interior cells from external attack, but also starve them through nutrient consumption. The invention further discovers that this conflict between protection and starvation is resolved through emergence of long-range metabolic codependence between peripheral and interior cells. As a result, biofilm growth halts periodically, increasing nutrient availability for the sheltered interior cells. This collective oscillation in biofilm growth benefits the community in the event of a chemical attack. The invention further provides that oscillations support population-level conflict resolution by coordinating competing metabolic demands in space and time, suggesting new strategies to control biofilm growth.

In certain embodiments, the invention provides a method of controlling and/or treating biofilms comprising the steps of: a) generating metabolic codependence of the growth of peripheral cells of the biofilms; b) establishing successful development of metabolic codependence within the biofilm community; c) inducing unrestricted growth of biofilm peripheral cells that lethally starves the protected interior cells; and d) eliminating accessible peripheral cells, or a combination thereof.

In certain embodiments, the growth of the periphery cells is promoted by supplying a media comprising minimal salts supplemented with glycerol and glutamate. In certain embodiments, the glycerol and glutamate is about 0.5% weight/volume, respectively. The media is non-toxic to humans. In other embodiments, the growth of the periphery cells is promoted by manipulating the activity of metabolic enzyme glutamate dehydrogenase (GDH).

In certain embodiments, the biofilm is exposed to ammonium, ammonia gas, or a combination thereof, in a sufficient amount and for a sufficient period of time to promote the growth of peripheral cells that is independent from nutrients, such as ammonium, provided by interior cells of the biofilms. The accessible peripheral cells are then treated and eliminated with a solution comprising an effective amount of a biofilm control substance, such as an antibiotic or toxic chemical. In certain embodiments, the toxic chemical is hydrogen peroxide, e.g. 1-4% hydrogen peroxide. In certain embodiments, the biofilm is a microbial colony comprising Bacillus subtilis.

In certain embodiments, the invention provides a method of treating or controlling biofilms in an oil pipeline or a pipeline of a water supply system, such method comprising: a) generating metabolic codependence of the growth of peripheral cells of the biofilms on ammonium produced by protected interior cells within the biofilms; b) establishing successful development of metabolic codependence of fluctuations in glutamate consumption within the biofilm; c) inducing unrestricted growth of biofilm peripheral cells that consume the nutrients for interior cells, causing the interior cells starved to death; d) eliminating accessible peripheral cells; and e) verifying treatment effectiveness.

Biofilms that form in pipelines can severely clog fluid flow. The invention provides that expose the pipeline, and thereby the biofilms within, to either ammonia gas or ammonium solution for two to six hours (depending on degree of contamination). Supplementation with ammonium or ammonia makes growth of peripheral cells independent from metabolites provided by interior cells. As a result, persistent growth of peripheral cells consumes the nutrients before they reach interior cells, lethally starving them. Once the protected interior cells are starved to death, the remaining peripheral cells can easily be eliminated by applying 1-4% hydrogen peroxide solution (again depending on level of contamination). Since interior cells are already dead, killing of peripheral cells after ammonium treatment minimizes damage to piping, while maximizing killing efficacy. After treatment is complete, samples of fluid flown through the pipeline are used to assess the number of remaining viable bacteria within the pipeline.

The invention methods and materials described herein can be used in a variety of applications including, but not limited to: (1) clinical Medicine, for the treatment or control of biofilm infections (2/3 of all clinical infections are caused by biofilms); (2) dental medicine, for the treatment of biofilms that form on teeth; (3) treatment of Medical equipment known to be susceptible to colonization by biofilms; (4) the food industry, to control biofilm contamination during food processing and storage; (5) the oil industry, where biofilms are known to clog up oil pipelines; and (6) water supply systems (as indicated by a report by the EPA, biofilms are a major threat to pipelines delivering drinking water systems).

These and other aspects of the present invention will be apparent to those of ordinary skill in the art in the following description, claims and drawings.

### BRIEF DESCRIPTION OF THE DRAWINGS

This patent application file contains at least one drawing executed in color. Copies of this patent application with color drawing(s) will be provided by the Office upon request and payment of the necessary fee.

FIGS. 1a-1h illustrate that biofilms grown in microfluidic devices show oscillations in colony expansion. FIG. 1a. Biofilms reconcile opposing demands for protection from external challenges and access to nutrients (gradient indicated in gray). FIG. 1b. Schematic of the microfluidic device used throughout the study described herein. Direction of media flow is indicated by the arrows. FIG. 1c, Phase contrast image of a biofilm growing in the microfluidic device. The arrow indicates the region of interest in panel FIG. 1(d). FIG. 1d. Filmstrip of a radius of the biofilm over time shows a pause in colony expansion. This film strip represents one cycle of biofilm oscillations, indicated by the shaded region in panel FIG. 1e. Scale indicates 5 μm. FIG. 1e. Growth rate over time shows persistent oscillations in colony expansion. FIG. 1f. Histogram of the average period of oscillations for each colony (n=63 colonies, mean=2.5 hours, s.d.=0.8 hours). The cell replication time is approximately 3.4 hours under the conditions described below in the method section. FIG. 1g. Growth rate as a function of colony diameter (which increases in time) shows that early colony growth does not exhibit oscillations. The orange line indicates the diameter (˜600 μm) at which this colony initiates oscillations. FIG. 1h. Histogram of the diameter at which a colony begins to oscillate (n=53 colonies, mean=576 s.d.=85 μm).

FIGS. 2a-2g illustrate that biofilm growth depends specifically on extracellular ammonium availability. FIG. 2a. Colony growth in MSgg medium depends on the production of glutamine from externally supplied glutamate and self-produced or scavenged ammonium. Glutamine limitation was monitored using YFP expressed from the nasA promoter, which is activated upon glutamine limitation²¹. FIG. 2b. Addition of 1 mM glutamine (shading) represses expression from the PnasA-YFP reporter (black), but does not affect expression from a constitutive reporter (Phyperspank-CFP+1 mM IPTG, gray). FIG. 2c. Growth area before and after addition of 1 mM glutamine to an oscillating colony. FIG. 2d. Of the two nutrients required for glutamine production, externally supplied glutamate is most abundant in the biofilm periphery, while biofilm-produced ammonium is most abundant in the biofilm interior. FIG. 2e. Maximum intensity projection over one period of a colony oscillation, which shows regions of growth (white) and no growth (black). Scale bar represents 100 μm. FIG. 2f. Growth area of an oscillating colony before and after addition of 30 mM glutamate (shading). FIG. 2g. Growth area of an oscillating colony before and after addition of 1 mM ammonium (shading).

FIGS. 3a-3h illustrate that mathematical modeling of a spatial metabolic feedback loop gives rise to oscillations consistent with experimental data. FIG. 3a. The production of ammonium in the interior is limited by and at the same time triggers the consumption of glutamate in the periphery, producing a delayed negative feedback loop. FIG. 3b. The excess glutamate not consumed by the biofilm periphery diffuses to the interior, where it can be converted into ammonium. The ammonium in turn enhances growth in the periphery and consequently reduces the supply of glutamate to the interior. Model predictions are shown in FIGS. 3c-3h: FIG. 3c. Biofilm growth over time. FIG. 3d. Glutamate concentration over time. FIG. 3e. Ammonium concentration over time. FIG. 3f. Colony growth before and after glutamine addition (indicated by the shading). FIG. 3g. Colony growth before and after addition of glutamate (indicated by the shading). FIG. 3h Colony growth before and after addition of ammonium (indicated by the shading).

FIGS. 4a-4i illustrate that metabolic codependence between interior and peripheral cells gives rise to oscillations that make the colony more resilient to external attack. FIG. 4a. Visual representation of the predicted outcome of an external attack on biofilm growth. FIG. 4b. Phase contrast merged with cell death marker (cyan, 1 μM Sytox) images of a wild type biofilm region shows cell death with and without challenge by 2% w/w H₂O₂. Scale bar represents 50 μm. FIG. 4c. In the same biofilm, difference images (white regions indicate cell growth) show wild type growth with and without challenge by H₂O₂. FIG. 4d. Overexpression of glutamate dehydrogenase (GDH) promotes more production of ammonium from glutamate. FIG. 4e, Experimental (top) and modeling results (bottom) of GDH overexpression (induced with 1 mM IPTG, indicated by shading). FIG. 4f. Phase contrast merged with cell death marker (cyan, 1 μM Sytox) images of a colony overexpressing GDH with and without challenge by H₂O₂. FIG. 4g. In the same biofilm, difference images show cell growth during GDH overexpression alone, and with challenge by H₂O₂. FIG. 4h. Quantification of total biofilm growth rate in wild type (upper, n=4 colonies) and GDH overexpression (lower, n=3 colonies) strains upon challenge with H₂O₂. Error bars represent standard deviations. Modeling data are shown as an inset for each strain. FIG. 4i. Codependence between interior and peripheral cells exhibited in a wild type strain results in a growth strategy that sustains the viability of interior cells, while independence enforced by a GDH overexpression strain results in starvation of interior cells and reduced resilience to external attack.

FIGS. 5a-5b illustrate characterization of biofilm growth oscillations. FIG. 5a, (Top) Growth rate over time of an oscillating colony. (Bottom) The pressure that drives media flow in the microfluidic chamber is constant over time. FIG. 5b. (Top) Growth rate of an oscillating colony. (Bottom) Period of each oscillation cycle, measured peak to peak. The error bars (±20 min) are determined by the imaging frequency (1 frame/10 min). The period slightly increases over time (see also FIG. 10f).

FIGS. 6a-6b illustrate roles of carbon and nitrogen in biofilm growth oscillations. FIG. 6a. Effect of increasing carbon (glycerol) or nitrogen (glutamate) availability on the oscillations. While increasing glutamate by 5 times of the normal MSgg levels leads to quenching of the oscillation, increasing glycerol by 5 times does not. FIG. 6b. Colony growth of mutant strain with rocG deletion. B. subtilis NCIB 3610 has two glutamate dehydrogenases (GDH), rocG and gudB. While gudB is constitutively expressed, rocG expression is subject to carbon catabolite repression¹⁸. The oscillatory growth of the rocG deletion strain indicates that carbon-source dependent regulation of rocG expression is not required for biofilm oscillations.

FIGS. 7a-7c illustrate fourier transform of biofilm growth rates before and after addition of 1 mM glutamine (FIG. 7a), 1 mM ammonium (FIG. 7b), and 1 mM IPTG (FIG. 7c) to induce Phyperspank-RocG. The error bars show standard deviations (n=3 colonies for each condition). The arrows indicate the frequency of oscillations for each condition before perturbation (left) and the lack of oscillations after perturbation (right).

FIGS. 8a-8b illustrate measurements of cell growth within oscillating biofilms. FIGS. 8a. (Top) Visual representation of the method described below. Growth is represented by white pixels, and lack of growth is indicated by black pixels. (Middle) Film strip and (bottom) growth area over time of an oscillating colony. Dashed lines show the position of each image on the time trace. Scale bar represents 100 μm. FIGS. 8b. (Top left) schematic of a biofilm. (Top right) high magnification phase contrast image of biofilm periphery focused at the bottom layer of cells. (Bottom panel) time traces depicting elongation rates of single cells in gray. The single cell time trace is pointed for the cell outlined in the top right panel. The periodic slowdown of the growth of individual peripheral cells is responsible for the observed periodic reduction in biofilm expansion.

FIGS. 9a-9b illustrate effects of external ammonium on biofilm development. FIG. 9a. Addition of external ammonium (shading, 1 mM) represses expression from the PnasA-YFP reporter (black), but does not affect expression from a constitutive reporter (Phyperspank-CFP+1 mM IPTG, gray). FIG. 9b. Removal of external ammonium (shading, 13 mM) causes halting of colony growth.

FIGS. 10a-10h illustrate a mathematical model of biofilm growth. FIG. 10a. The model describes the dynamics of two cell populations in a biofilm, interior and peripheral. As the biofilm grows, there is a constant distance between the interior population and the biofilm edge. FIGS. 10b-10e. Bifurcation diagrams showing systematic analysis on the effects of external glutamine, external glutamate, ammonium uptake, and GDH overexpression respectively. The gray lines correspond to the extrema of oscillations in peripheral glutamate (stable limit cycle). The solid black line denotes stable fixed point. The dashed black line corresponds to an unstable fixed point. The vertical gray lines highlight the state of the system for each nutrient addition experiment shown in FIGS. 3a-3h above. FIG. 10f. Model prediction of oscillation period as function of interior cell fraction in the whole biofilm. FIGS. 10g-10h, Sensitivity analysis of oscillation period and modulation depth to changes in model parameters. Modulation depth is defined as the amplitude of the oscillations divided by the mean value. Gray color denotes parameter regions where the system does not oscillate.

FIGS. 11a-11b illustrate a temporal profile of cell death within an oscillating biofilm. FIG. 11a. Colony growth rate. FIG. 11b. Average fluorescence intensity of a cell death marker (Sytox Green, 1 μM, Life Technologies) from the same colony shown in FIG. 11a.

FIG. 12 illustrates an effect of external attack with hydrogen peroxide (H₂O₂, 0.15% v/v) or chloramphenicol (CM, 5 μg/ml). (Top) cell death shown by Sytox Green (1 μM). (Middle and bottom) colony growth shown by image differencing (see FIG. 8a above). Scale bar represents 100 μm. The white dashed lines indicate colony edge.

FIG. 13 illustrates an effect of GDH induction on cell growth. Wild type and Phyperspank-RocG (uninduced or induced with 10 mM IPTG) strains were grown in liquid culture (MSgg medium, 30° C.). Cell generation times were measured using OD₆₀₀. Error bars show standard deviations (n=3 replicates).

FIGS. 14a-14e illustrate growth rate oscillations persist in various mutant strains. FIG. 14a. opp operon deletion (deficient in quorum sensing). FIG. 14b. comX deletion (deficient in quorum sensing). FIG. 14c. tapA operon deletion (extracellular matrix component deletion). FIG. 14d. tapA operon overexpression (Phyperspank-tapA operon, 1 mM IPTG). FIG. 14e. hag deletion (deficient in swimming and swarming). These results show that the corresponding genes and processes are not required for biofilm oscillations.

### DETAILED DESCRIPTION OF THE INVENTION

Disclosed are methods and materials that pertain to the control/treatment of structured microbial colonies known as biofilms. Specifically, having discovered that cells within a community both cooperate and compete with each other for nutrient resources, and exhibit metabolic codependence, the inventors developed a novel approach to killing biofilms more effectively, by first killing the most protected interior cells. Potential applications include treatment of clinical infections/dental biofilms, biofilm control on medical equipment and food, and in oil or water supply pipelines.

In the invention, supplying the biofilm with ammonium (or ammonia gas) promotes unrestricted growth of cells that occupy the biofilm periphery. Nutrient consumption by peripheral cells leads to lethal starvation of interior cells, those that are the most protected and most likely to survive a chemical or antibiotic treatment. The invention enables first eliminating the protected interior cells, thus leaving behind only the most sensitive and easy to access and eradicate cells at the biofilm periphery. This treatment can also be applied by manipulating the activity of the metabolic enzyme glutamate dehydrogenase (GDH).

In certain embodiments, the data presented in the invention reveal that intracellular metabolic activity within biofilms is organized in space and time, giving rise to codependence between interior and peripheral cells. Even though bacteria are single-celled organisms, the metabolic dynamics of individual cells are regulated in the context of the community. This metabolic codependence in turn gives rise to collective oscillations that emerge during biofilm formation and promote the resilience of biofilms against chemical attack. The community-level oscillations also support the ability of biofilms to reach large sizes, while retaining a viable population of interior cells. Specifically, periodic halting of peripheral cell growth prevents complete starvation and death of the interior cells. This overcomes the colony size limitation for a viable biofilm interior that would otherwise be imposed by nutrient consumption in the biofilm periphery. Metabolic codependence in biofilms therefore offers an elegant solution that resolves the social conflict between cooperation (protection) and competition (starvation) through oscillations.

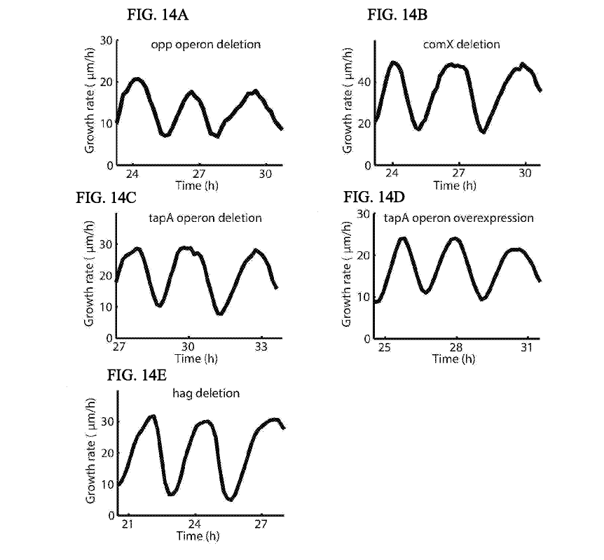

The invention discovered biofilm oscillations. While cellular processes such as swarming or expression of extracellular matrix components are not required for the observed biofilm oscillations (FIGS. 14a-14e), it may suggest that such cellular processes are influenced by oscillatory dynamics²⁹. Also, metabolic codependence may also arise in other biofilm-forming species. Other metabolic branches where metabolites can be shared among cells could also give rise to oscillations in biofilm growth.

The invention further suggests strategies to cope with the intriguing resilience of biofilms in the face of environmental stresses, such as antibiotic exposure. In particular, the invention shows that straightforward application of stress (such as H₂O₂ or chloramphenicol) to the biofilm counterintuitively promotes growth, effectively rejuvenating the biofilm. Death of the colony periphery relieves the repression on the growth of interior cells, allowing them to regenerate a new biofilm periphery and interior. In contrast, manipulation of the metabolic codependence yields a more effective approach to control biofilm formation. Specifically, promoting continuous growth of peripheral cells can starve the biofilm interior, leaving behind the exposed peripheral cells that can more easily be targeted by external killing factors. Therefore, the metabolically driven collective oscillations in biofilm expansion described in the invention not only reveal fundamental insights into the principles that govern formation of multicellular communities, but also suggest new strategies for manipulating the growth of biofilms.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Methods

Strains and Plasmids. All experiments were done using Bacillus subtilis NCIB 3610. The wild type strain was a gift from University of Maryland³⁰ and all other strains were derived from it and are listed in Table 1.

TABLE 1
List of strains used in this study

| Strain | Genotype | Source |
|---|---|---|
| Wild type | B. subtilis NCIB 3610 | 30 |
| P_{hyp}-CFP, P_{nasA}-YFP | amyE:: P_{Hyperspank}-cfp, | This study |
| | sacA:: PnasA-yfp (Sp^{R}, Cm^{R}) | |
| P_{hyp}-RocG | amyE:: P_{Hyperspank}-rocG (Sp^{R}) | This study |
| P_{hyp}-tapA operon | amyE:: P_{Hyperspank}-tapA operon (Sp^{R}) | This study |
| ΔtapA operon | tapA-sipW-tasA:: cat | This study |
| ΔoppA-D | oppABCD:: cat | This study |
| ΔcomX | comX:: cat | 14 |
| Δhag | hag:: cat | This study |
| ΔrocG | rocG:: kan | This study |

The pSac-CM-PnasA-yfp vector was a gift from California Institute of Technology. To make overexpression strains, polymerase chain reaction (PCR) was used to amplify the desired region from the wild type strain. The PCR product was then put under the Hyperspank IPTG inducible promoter, using the HindIII/SaII and the NheI sites within the pDR111 vector. The Hyperspank promoter controls overexpression of the gene through isopropyl-β-D-thiogalactopyranoside (IPTG) induction. Vectors for deletion strains were made in a similar way using the pER449 plasmid. All constructs were confirmed by direct sequencing and then integrated into the chromosome of the wild type strain by a standard one-step transformation procedure³¹. Chromosomal integrations were confirmed by colony PCR using the corresponding primers.

Growth conditions. The biofilms were grown using MSgg medium¹⁶. It contains 5 mM potassium phosphate buffer (pH 7.0), 100 mM MOPS buffer (pH 7.0, adjusted using NaOH), 2 mM MgCl₂, 700 μM CaCl₂, 50 μM MnCl₂, 100 μM FeCl₃, 1 μM ZnCl₂, 2 μM thiamine HCl, 0.5% (v/v) glycerol and 0.5% (w/v) monosodium glutamate. The MSgg medium was made from stock solutions on the day of the experiment, and the stock solution for glutamate was made each week.

Microfluidics. The CellASIC ONIX Microfluidic Platform and the Y04D microfluidic plate (EMD Millipore) were used. It provides unconventionally large chambers, allowing the formation of colonies containing millions of cells, yet still leaves room for media flow. Media flow in the microfluidic chamber was driven by a pneumatic pump from the CellASIC ONIX Microfluidic Platform, and the pressure from the pump was kept stable during the course of the oscillation. In most of the experiments, a pump pressure of 1 psi was used with only one media inlet open (there are 6 media inlets in the Y04D plate), which corresponds to a flow speed of ˜16 μm/s in the growth chamber.

On the day before the experiment, cells from −80° C. glycerol stock were streaked onto LB agar plate and incubated at 37° C. for overnight. The next day morning, a single colony was picked from the plate and inoculated into 3 ml of LB broth in a 50 ml conical tube, and then incubated in 37° C. shaker. After 2.5 hours of incubation, the cell culture was centrifuged at 2100 rcf for 1 min, and then the cell pellet was re-suspended in MSgg and then immediately loaded into microfluidics. After the loading, cells in the microfluidic chamber were incubated at 37° C. for 90 min, and then the temperature was kept at 30° C. for the rest of the experiment.

Time-Lapse Microscopy. The growth of the biofilms was recorded using phase contrast microscopy. The microscopes used were Olympus IX81 and IX83, and DeltaVision PersonalDV. To image entire biofilms, 10× lens objectives were used in most of the experiments. Images were taken every 10 min Whenever fluorescence images were recorded, the minimum exposure time was used that still provided a good signal-to-noise ratio.

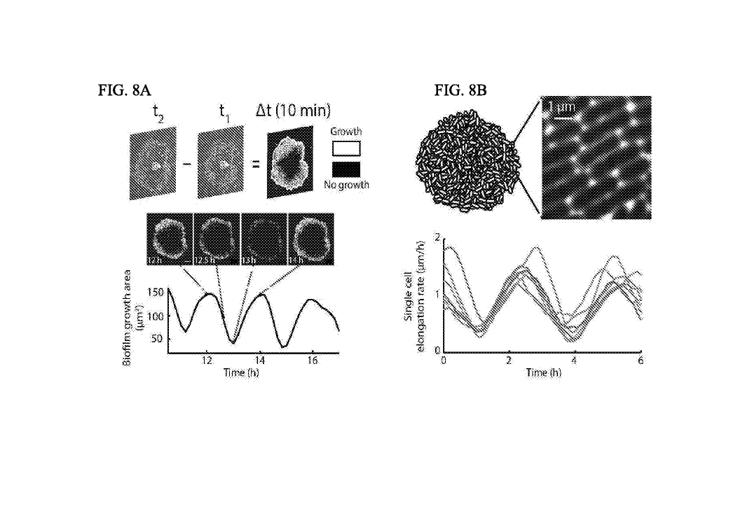

Biofilm growth rate. ImageJ (National Institutes of Health) and MATLAB (MathWorks) were used for image analysis. In house software was also developed to perform colony detection and quantification of colony expansion. Multiple methods of colony detection were used to ensure the accuracy of the analysis. To detect regions of expansion in a biofilm, image differencing was performed on snapshots of the biofilm from time-lapse microscopy videos. Specifically, the difference between two consecutive phase contrast images (taken 10 min apart) was calculated by finding the absolute difference between each pixel in each image. An image stack was then generated based on these results. The intensity values from the stack correlate with the expansion inside the biofilm. The growth area was determined by converting difference images to binary images and then measuring the area of the colony growth region (white pixels). To measure cell replication time, the length and division of individual cells were tracked in the biofilm periphery (FIG. 8b).

### Example 2

The Opposing Benefits of Growth and Protection During Biofilm Development

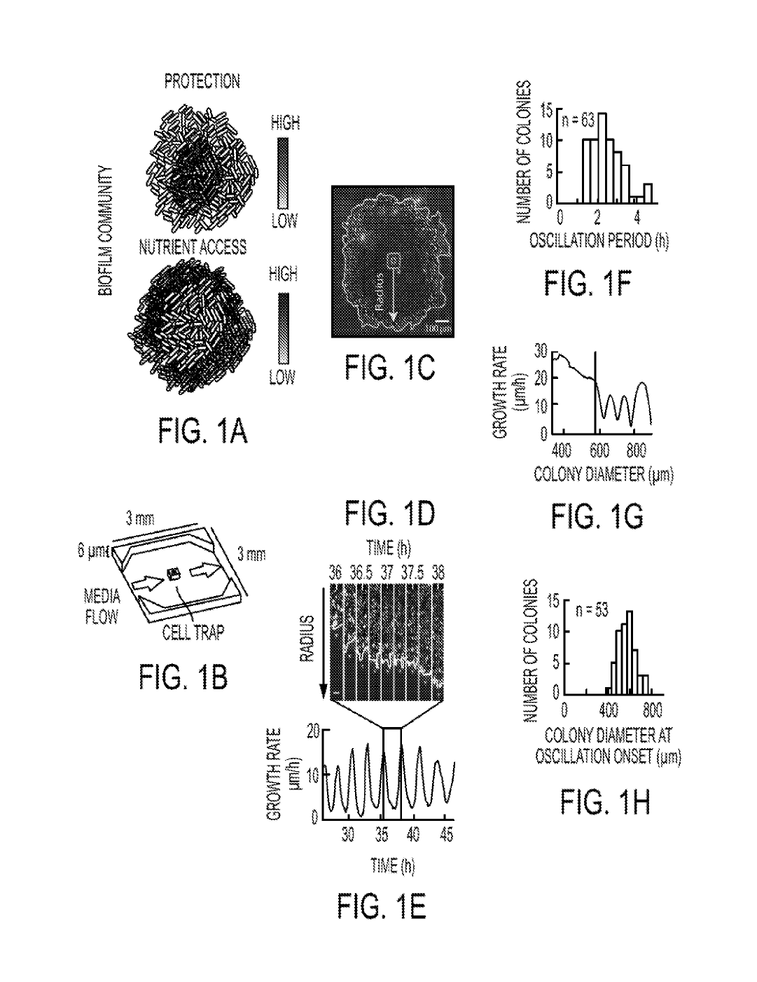

Bacterial biofilms⁷⁻¹⁰ were investigated to determine how the conflict between the opposing social behaviors of cooperation and competition could be resolved at the community level to increase overall fitness. Biofilms typically form under environmental stress conditions, such as nutrient limitation¹¹⁻¹³ . As these bacterial communities grow larger, the supply of nutrients to interior cells becomes limited due to an increase in nutrient consumption associated with the growth of multiple layers of cells in the biofilm periphery. Severe nutrient limitation for interior cells is detrimental to the colony, since the sheltered interior cells are critical to the survival of the biofilm community in the event of an external challenge. This defines a fundamental conflict between the opposing demands for biofilm growth and maintaining the viability of protected (interior) cells (FIG. 1a). The identification of possible mechanisms that ensure the viability of the protected interior cells is fundamental to understanding biofilm development^{14, 15}.

In order to directly investigate how Bacillus subtilis biofilms continue expanding while sustaining interior cells, the potentially complex three-dimensional problem was converted to a simpler two-dimensional scenario using microfluidics. Specifically, growth chambers that are unconventionally large in the lateral, x-y dimensions (3×3 mm) was used while confining biofilm thickness (z-dimension) to only a few micrometers (FIG. 1b). Therefore, biofilm expansion in this device is predominantly limited to two dimensions, creating a “pancake-like” configuration. In fact, biofilms often form in confined aqueous environments and thus this microfluidic chamber may better mimic those growth conditions¹¹⁻¹³. This experimental set-up is thus ideal to interrogate how biofilms can reconcile the opposing benefits of growth and protection during biofilm development.

### Example 3

### Oscillations in Biofilm Growth

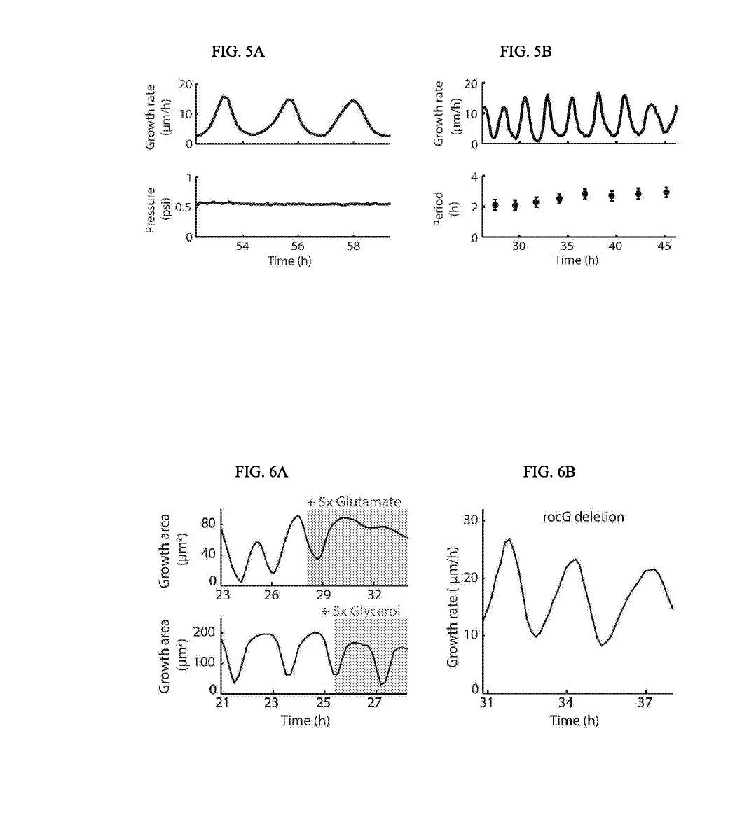

Unexpectedly, oscillations were observed in biofilm expansion despite constant media flow within the microfluidic device (FIG. 1c, FIG. 1d, and FIG. 5a). Specifically, biofilms exhibit periodic reduction in colony expansion that is self-sustained and can last for more than a day (FIG. 1e and FIG. 5b). The period of oscillations has a mean of 2.5±0.8 hours (s.d., n=63 colonies), which is less than the duration of the average cell replication time of 3.4±0.2 hours (s.d., n=21 cell cycles) under this growth condition (FIG. 1f). Moreover, oscillations only arise when the biofilm exceeds a certain colony size. In particular, quantitative measurements obtained from 53 individual biofilms indicate that oscillations emerge in colonies that exceed an average diameter of 580±85 μm (s.d., n=53 colonies), which corresponds to approximately one million cells (FIG. 1g, FIG. 1h). Together, these data show that oscillations arise during biofilm formation and are self-sustained.

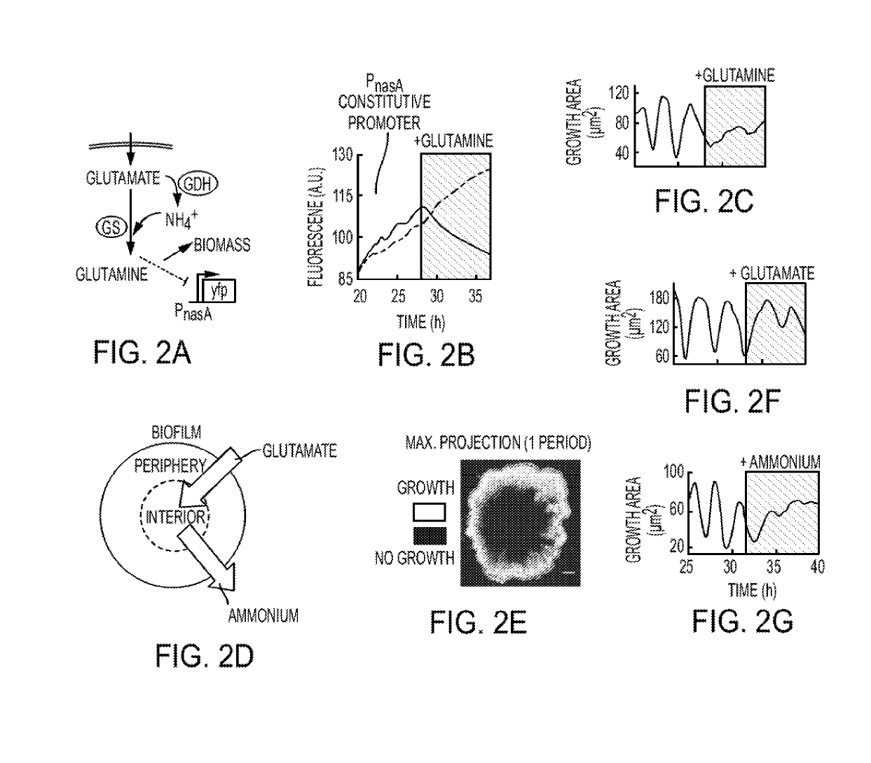

Given that biofilms typically form under nutrient limited conditions and bacterial growth is generally controlled by metabolism, metabolic limitation plays a key role in the observed periodic halting of biofilm expansion. In particular, after determining that carbon source limitation did not play an essential role in the oscillations (FIGS. 6a-6b), nitrogen limitation was focused on. The standard biofilm growth media (MSgg) used to study B. subtilis biofilm development contains glutamate as the only nitrogen source¹⁶. In most organisms including B. subtilis, glutamate is combined with ammonium by glutamine synthetase (GS) to produce glutamine, which is essential for biomass production and growth (FIG. 2a)¹⁷. Cells can obtain the necessary ammonium from glutamate through the enzymatic activity of glutamate dehydrogenase (GDH), expressed by the rocG or gudB genes in the undomesticated B. subtilis used in this study (FIG. 2a)¹⁸⁻²⁰.

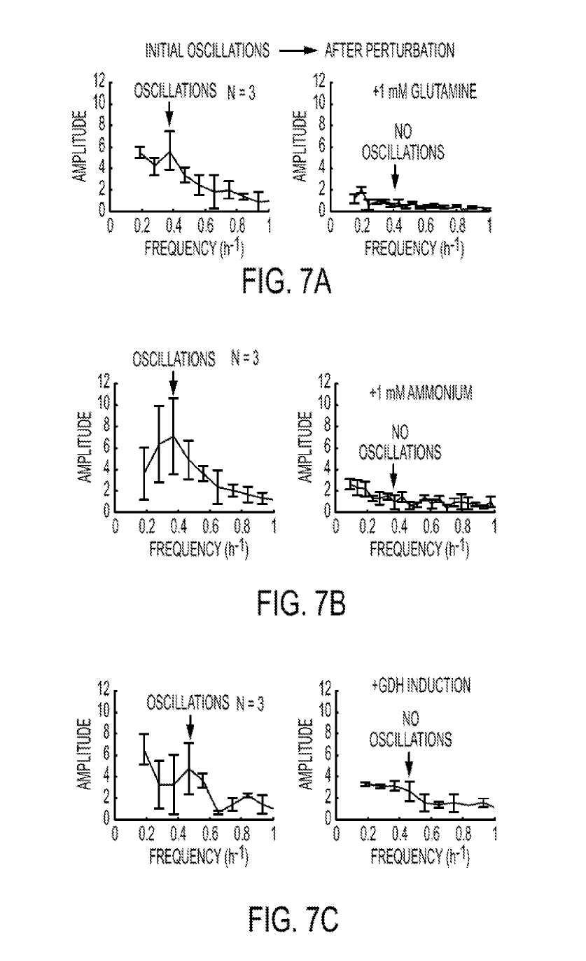

To determine whether biofilms experience glutamine limitation, expression of nasA, one of several genes activated in response to a lack of glutamine²¹, was measured. Results show that biofilms indeed experience glutamine limitation during growth. Specifically, supplementation of growth media directly with glutamine reduced nasA promoter expression, but did not affect expression of a constitutive promoter, confirming glutamine limitation within the biofilm (FIG. 2b). More strikingly, addition of exogenous glutamine eliminated periodic halting of biofilm growth (FIG. 2c and FIG. 7a). These findings suggest that glutamine limitation plays a critical role in the observed oscillations during biofilm expansion.

The synthesis of glutamine requires both glutamate and ammonium, therefore it was further investigated which of these substrates could be responsible for the observed glutamine limitation. Glutamate was provided in the media and was thus readily available to cells in the periphery of the biofilm. On the other hand, consumption of glutamate by peripheral cells was likely to limit its availability to cells in the biofilm interior (FIG. 2d). One suggestion is that oscillations in biofilm expansion could be due to periodic pausing of cell growth in the biofilm interior. Accordingly, it was determined whether interior or peripheral cells exhibited changes in growth. By tracking physical movement within the biofilm, it was uncovered that only peripheral cells grow and that oscillations in biofilm expansion therefore arise exclusively from periodic halting of peripheral cell growth (FIG. 2e, FIG. 8a). This finding was further confirmed by single cell resolution analysis that directly showed periodic reduction in the growth of peripheral cells (FIG. 8b). This surprising pausing of cell growth in the periphery, despite unrestricted access to glutamate, suggests that glutamate cannot be the limiting substrate for glutamine synthesis. Consistent with this finding, biofilm oscillations were not quenched by supplementation of the media with glutamate (FIG. 2f). Therefore, it is not glutamate, but ammonium that is the limiting substrate for glutamine synthesis in the biofilm periphery.

Since cells can self-produce ammonium from glutamate, it was also sought to determine how peripheral cells could experience periodic ammonium limitation despite a constant supply of glutamate in the media. It is well known that ammonium production is a highly regulated process that is dependent on the metabolic state of the cell and the ambient level of ammonium in the environment²². In particular, since ammonium is in equilibrium with ammonia vapor, which can freely cross the cell membrane and be lost to the extracellular media²³, the production of ammonium is known as a “futile cycle”. Cells therefore preferentially use extracellular (ambient) ammonium for growth, rather than producing their own²⁴⁻²⁶. Since peripheral cells are exposed to media flow, they are particularly susceptible to this futile cycle of ammonia loss.

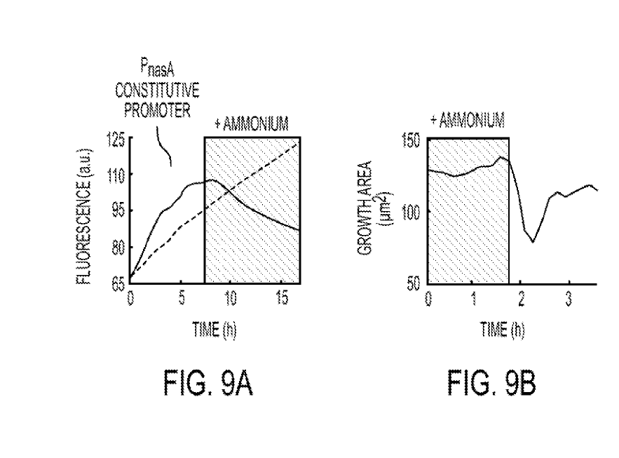

In this sense, since ammonium is not provided in the media, even if all cells produce ammonium, the biofilm interior is the major source for ambient ammonium (FIG. 2d). Consequently, growth of peripheral cells relies on ammonium produced within the biofilm. To confirm this theory, the media was supplemented with 1 mM ammonium, which eliminated the periodic halting in biofilm expansion (FIG. 2g, FIG. 7b, and FIG. 9a). When additional ammonium was suddenly removed from the media, growth in the biofilm periphery halted (FIG. 9b). These findings indicate that peripheral cells preferentially rely on extracellular ammonium produced within the biofilm for their growth.

### Example 4

### Metabolic Codependence between the Biofilm Periphery and Interior

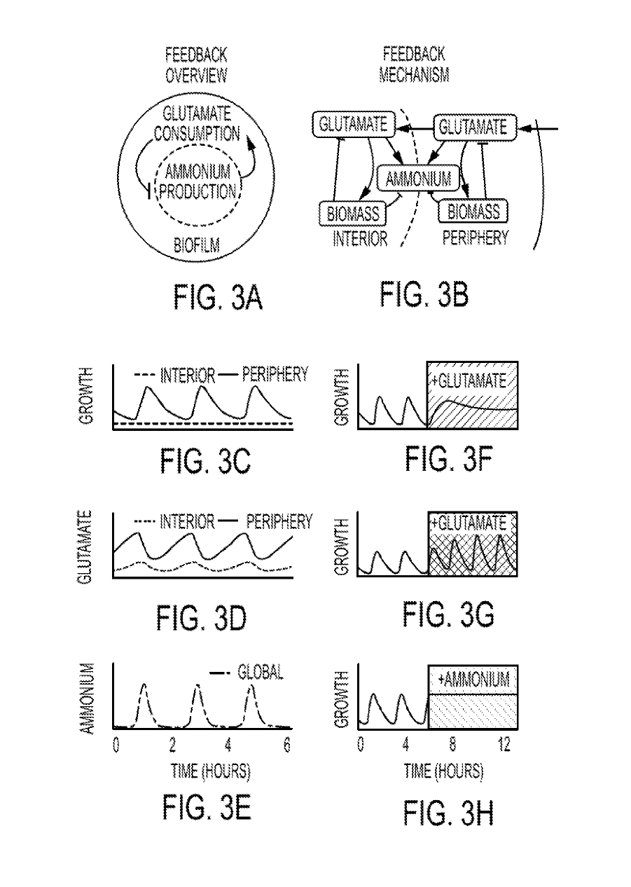

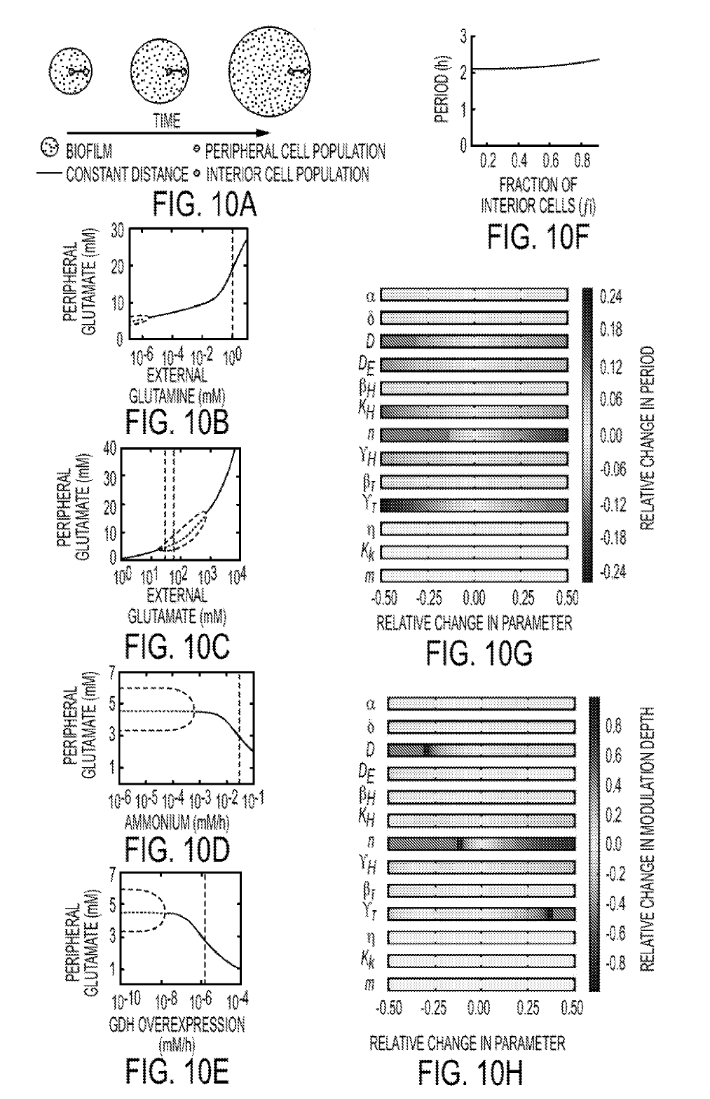

The results described above suggest that ammonium limitation for peripheral cells arises due to glutamate limitation for interior cells. Specifically, persistent consumption of glutamate by peripheral cells can deprive the interior cells of the necessary glutamate for ammonium production. In order to explore this nontrivial finding, a mathematical modeling is suggested to develop a conceptual framework and generate experimentally testable predictions. The mathematical model describes separately the metabolic dynamics of interior and peripheral cells and the metabolite exchange between them, where the distinction of the two subpopulations depends on nutrient availability. The model consists of two main assumptions (FIG. 3a): First, consumption of glutamate during growth of peripheral cells deprives interior cells of this nutrient and thus inhibits ammonium production in the biofilm interior. Second, the growth of peripheral cells depends predominantly on ammonium that is produced by metabolically stressed interior cells. A model based on these two simplifying assumptions (FIG. 3b) generates oscillations consistent with the experimental observations (FIG. 3c-3e) and reproduces the effects of supplementing the media with glutamine, glutamate and ammonium (FIG. 3f-h, FIGS. 10a-10h, see EXAMPLE 6 below for modeling). The model also accounts for the observed slight increase of the oscillation period by considering an increase in the ratio of interior to peripheral cells over time (FIGS. 5b and 10f). Therefore, this mathematical model shows that periodic halting in biofilm growth can result from metabolic codependence between cells in the biofilm periphery and interior that is driven by glutamate consumption and ammonium production, respectively.

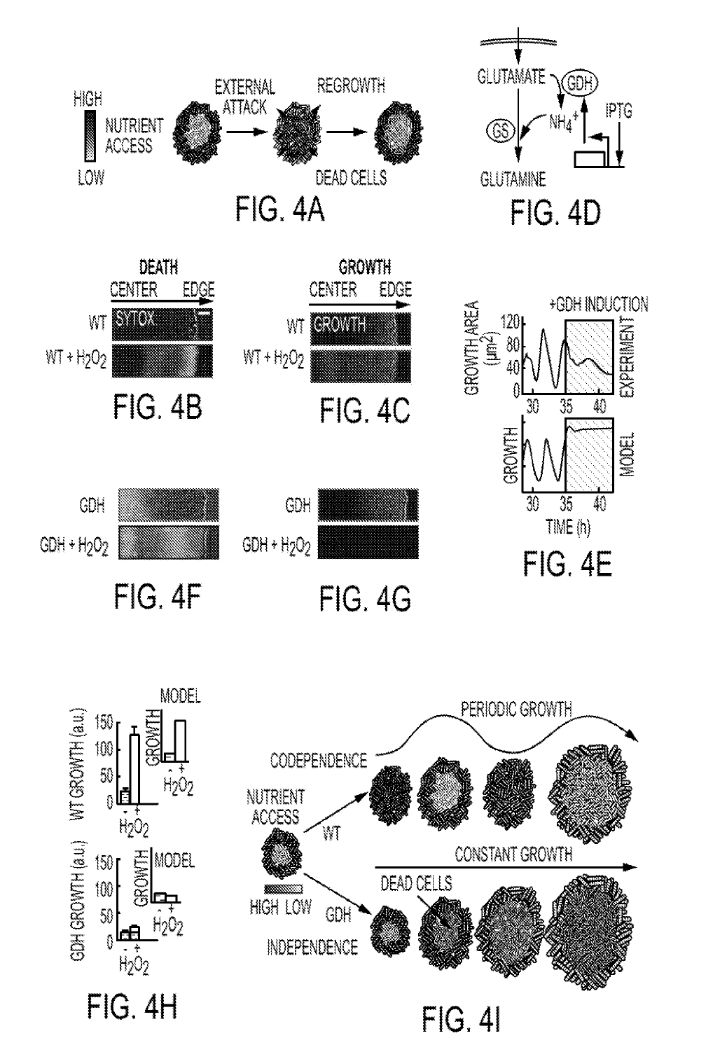

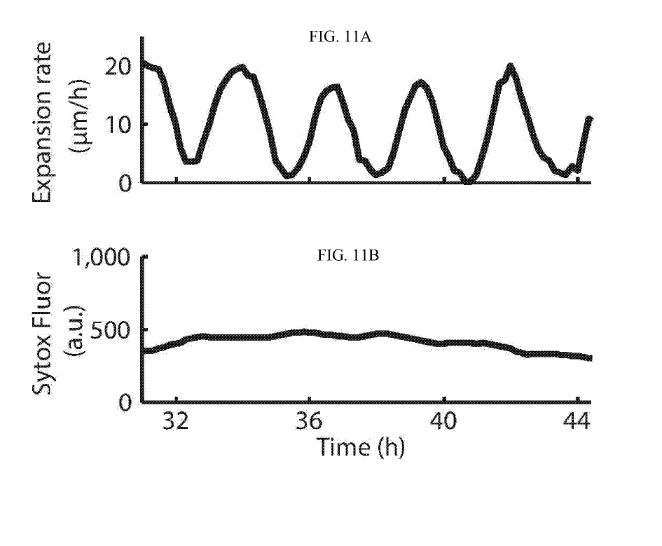

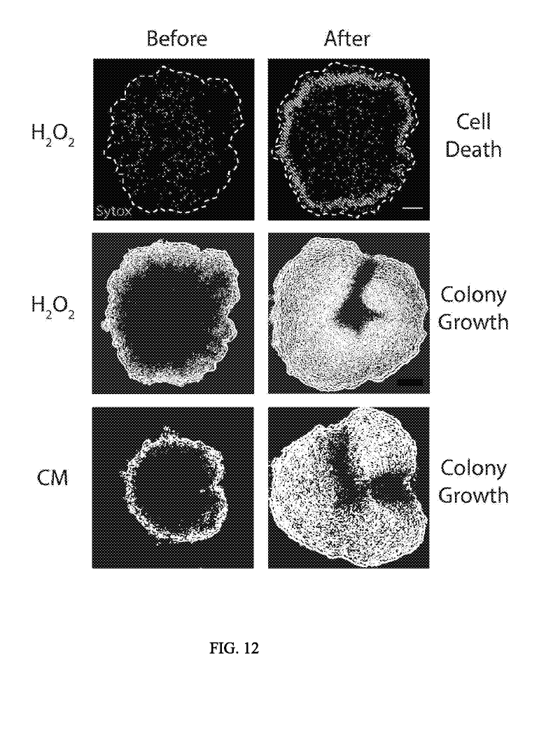

The metabolic codependence between interior and peripheral cells gives rise to the surprising findings that external attack could promote growth within the biofilm. Specifically, killing of peripheral cells eliminates their glutamate consumption, which increases glutamate availability in the biofilm and thereby promotes growth of interior cells (FIG. 4a). Accordingly, cell death and growth were measured within oscillating biofilms (FIG. 4b, top and FIGS. 11a-11b). When the biofilm was exposed to media containing hydrogen peroxide (H₂O₂), increased cell death was observed predominantly in the biofilm periphery (FIG. 4b, bottom and FIG. 12). Death of peripheral cells led to growth of interior cells (FIG. 4c and FIG. 12). To verify that this response is not uniquely triggered by H₂O₂, biofilms was exposed to the antibiotic chloramphenicol and again growth of interior cells was observed (FIG. 12). These findings further support that glutamate consumption by peripheral cells limits its availability in the biofilm.

### Example 5

### The Benefit of Biofilm Oscillations

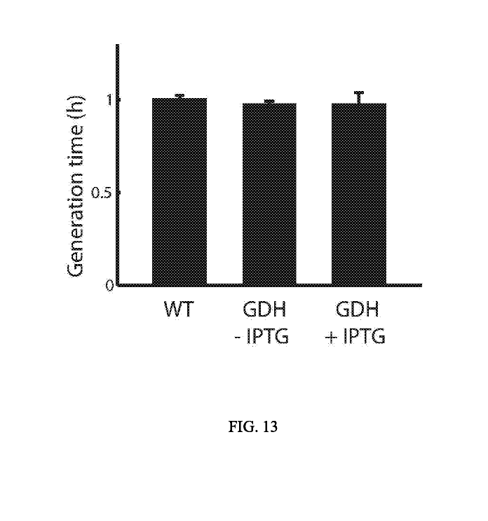

The mathematical model also assumes that glutamate starvation of the biofilm interior reduces the production of ammonium that can support peripheral cell growth. This assumption provokes the question as to why peripheral cells do not simply overcome their dependence on extracellular ammonium by increasing intracellular production^{27, 28}. To address this question, a strain was constructed that contains an inducible copy of the GDH gene rocG (FIG. 4d). It was confirmed that GDH overexpression was not toxic to individual cells and did not affect their growth rate (FIG. 13). In contrast, the induction of GDH expression in the biofilm quenched growth oscillations (FIG. 4e and FIG. 7c) and resulted in high levels of cell death in the colony interior (FIG. 4f, top). This result explains why peripheral cells do not appear to utilize the simple strategy of overcoming their dependence on extracellular ammonium: such a strategy would result in the continuous growth of peripheral cells, starving and ultimately causing the death of sheltered interior cells within the biofilm. Periodic halting of peripheral cell growth due to extracellular ammonium limitation thus promotes the overall viability of the biofilm.

The ability of the biofilm to regenerate itself in the event of an external attack suggested that killing the biofilm interior first is a more effective strategy for biofilm extermination. Accordingly, the GDH overexpression strain was exposed to hydrogen peroxide and growth and death were again measured. As described above, GDH induction causes death of interior cells. Exposing the GDH overexpression strain to hydrogen peroxide resulted in more effective global killing throughout the biofilm (FIGS. 4f and 4g, bottom). While in the wild-type biofilm interior cells begin to grow in response to an external attack, metabolic independence between interior and peripheral cells in the GDH strain interferes with this defense mechanism (FIG. 4h). This outcome is also consistent with modeling predictions (FIG. 4h, inset). Oscillations in biofilm growth that are driven by metabolic codependence thus promote the resilience of the biofilm community by sustaining the viability of the sheltered interior cells that are most likely to survive in the event of an environmental stress (FIG. 4i).

### Example 6

### Mathematical Model of Metabolic Codependence

### Model Description

The dynamics of biofilm growth were described in terms of two distinct populations, corresponding to the interior and the periphery of the biofilm. The two populations are assumed to be located in a moving frame of reference as the biofilm grows, so that they are always located at the same distance from the physical edge of the biofilm (FIG. 10a).

The metabolic state of the biofilm is determined by the following quantities: 1) The concentrations of glutamate in the biofilm interior (Gᵢ) and in the periphery (Gₚ); 2) the concentration of ammonium (A), which is assumed to be equal for the two populations due to its fast diffusion; 3) The concentration of active glutamate dehydrogenase (GDH) in the interior cells (Hᵢ); and 4) the rate of biomass production, which is assumed to be given by the concentrations of housekeeping proteins (such as ribosomal proteins) in the interior (rᵢ) and in the periphery (rₚ). The dynamics of these state variables are described by the following set of ordinary differential equations:

$$\frac{dA}{dt} = α {G}_{i} {H}_{i} - {δ}_{A} A ( {r}_{i} + {r}_{p} )$$

$$\frac{dGi}{dt} = D ( {G}_{p} - {G}_{i} ) - α {G}_{i} {H}_{i} - {δ}_{G} {G}_{i} {r}_{i}$$

$$\frac{{dG}_{p}}{dt} = D ( {G}_{i} - {G}_{p} ) + {D}_{E} ( {G}_{E} - {G}_{p} ) - {δ}_{G} {G}_{p} {r}_{p}$$

$$\frac{{dH}_{i}}{dt} = {β}_{H} \frac{{G}_{i}^{n}}{{K}_{H}^{ n} + {G}_{i}^{ n}} - {γ}_{H} {H}_{i}$$

$$\frac{{dr}_{i}}{dt} = {β}_{r} {AG}_{i} - {γ}_{r} {r}_{i}$$

$$\frac{{dr}_{p}}{dt} = {β}_{r} {AG}_{p} - {γ}_{r} {r}_{p}$$

The terms in the equations are interpreted as follows:
αGᵢHᵢ: ammonium production from glutamate, catalyzed by the enzyme GDH (FIG. 2a)
δ_{A}A(rᵢ+rₚ): ammonium consumption by interior and peripheral cells
δ_{G}Gᵢrᵢ and δ_{G}Gₚrₚ: glutamate consumption by interior and peripheral cells, respectively
D(Gₚ−Gᵢ): glutamate diffusion between peripheral and interior regions
D_{E}(G_{E}−Gₚ): glutamate diffusion between the environment and the periphery of the biofilm

$${β}_{H} \frac{{G}_{i}^{n}}{{K}_{H}^{ n} + {G}_{i}^{ n}} \text{:}$$

GDH activation in the interior cells
γ_{H}Hᵢ: GDH deactivation in the interior cells
βᵣAGᵢ and βᵣAGₚ: production of housekeeping proteins in the interior and peripheral cells, respectively
γᵣrᵢ and γᵣrₚ: degradation of housekeeping proteins in interior and peripheral cells, respectively

The following assumptions were made:
Peripheral cells rely on ammonium synthesized by interior cells. As a simplification, only the interior cells were assumed to have active GDH.
Activation of GDH depends on the glutamate availability. Specifically, Hᵢ is reduced when the concentration of available glutamate (Gᵢ) is below a given threshold. This can be due to explicit regulatory interactions or simply as a consequence of the slowdown of cellular processes in the absence of nutrients.
Consumption of ammonium and glutamate depends on the metabolic activity of the cell. The higher the concentration of housekeeping proteins—a proxy for the metabolic state of the cell—the faster the consumption of nutrients.
The production of housekeeping proteins increases with the concentrations of glutamate and ammonium.

In order to extract from the model the population expansion, which can be measured experimentally, we consider that the dynamics of the cell density ρ p of the two populations are given by:

$$\frac{d {ρ}_{i , p}}{dt} = η {r}_{i , p} {ρ}_{i , p} ( 1 - \frac{{ρ}_{i , p}}{K ( {G}_{i , p} )} ) - {λ}_{i , p} {ρ}_{i , p}$$

The first term in the right-hand side is a logistic-growth term, where the maximal growth rate is considered to be proportional to the concentrations of housekeeping proteins rᵢ and rₚ. Additionally, it was assumed that the carrying capacity K depends on the concentration of glutamate:

$$K ( G ) = \frac{{G}^{m}}{{K}_{k}^{ m} + {G}^{ m}}$$

Thus K (G) varies between 0 and 1 depending on whether glutamate concentration is below or above a given threshold, denoted as Kₖ. Note that the cell density ρ_{i,p} defined here is relative to the carrying capacity, therefore, both K and ρ are dimensionless.

The logistic-growth term in the density equation shown above describes the standard birth/death processes that occur in an unmoving bacterial population. In this system, however, the peripheral cells are always expanding into the open area outside of the biofilm. This fact is represented by adding an effective decay term, −λ_{i,p}ρ_{i,p} in the density equation of the expanding population (i.e. the peripheral population for all situations considered, except in the case of chemical attack, where the peripheral population is eradicated and consequently the interior cells can expand). This decay term accounts for the effective loss of cells undergone locally by the biofilm front as it expands (in the moving reference frame) into the cell-free area surrounding it.

Given the above-described dynamics for the cell densities, the growth rate (measured experimentally as the area of non-zero local motion within the biofilm) is given by the logistic term, since this is the only term related to actual growth of the population:

$${μ}_{i , p} = η {r}_{i , p} {ρ}_{i , p} ( 1 - \frac{{ρ}_{i , p}}{K ( {G}_{i , p} )} )$$

### Addition of Glutamine to the Media

Glutamine is synthesized by glutamine synthase (GS) in the cell, and it also regulates the activity of GS through negative feedback²⁷. Therefore, external addition of glutamine reduces GS activity, and consequently lowers its consumption of ammonium and glutamate (used to synthesize glutamine) Additionally, it is assumed that glutamine inhibits either directly or indirectly GDH activity, affecting the production of ammonium from glutamate. This is implemented in the model as non-competitive inhibition on the parameters α and δ. Specifically, the effective α̅ and δ̅ are given by:

$$\overline{α} = \frac{α}{\frac{[ Gln ]}{{K}_{α}} + 1} , \overline{{δ}_{A , G}} = \frac{{δ}_{A , G}}{\frac{[ Gln ]}{{K}_{δ}} + 1}$$

FIG. 3f shows the model prediction: in agreement with the experimental observations, external addition of glutamine leads to the quenching of oscillation. A systematic analysis of the effect of glutamine addition is shown in FIG. 9b, where a bifurcation diagram of the peripheral glutamate concentration with respect to the added glutamine concentration is shown.

### Addition of Glutamate to the Media

The concentration of glutamate in the external medium is explicitly defined in the model by the parameter G_{E}. Thus, supplementation with additional glutamate is represented by simply increasing the value of G_{E}.

FIG. 3g shows the model prediction: consistent with the experimental observations, a moderate increase in external glutamate does not eliminate the oscillations. A systematic study also shows that further increasing glutamate leads to quenching of oscillations (FIG. 10c).

### Addition of Ammonium to the Media

The concentration of ammonium is explicitly represented in the model with the variable A, and addition of ammonium to the media can be represented as an additional creation term (α₀) in the ammonium equation:

$$\frac{dA}{dt} = α {G}_{i} {H}_{i} - {δ}_{A} A ( {r}_{i} + {r}_{p} ) + {α}_{0}$$

FIG. 3h shows the model prediction: in agreement with the experiments, externally adding ammonium quenches oscillation. It was also systematically explored the effect of different ammonium concentrations through a bifurcation diagram of the system with respect to α₀ (FIG. 10d).

### Overexpression of GDH in Cells

It was also investigated the effects of overexpressing GDH in the biofilm. The overexpression is implemented in the model by an additional creation term β₀ into the equation for GDH (Hᵢ). Furthermore, since the overexpression is applied throughout the entire biofilm, active GDH was included for the peripheral cells (Hₚ), and consequently the production of ammonium from those cells. To that end, the differential equations for A, Gₚ and Hᵢ are modified as shown below, and an equation for GDH in the peripheral cell population (Hₚ) is also added:

$$\frac{dA}{dt} = α {G}_{i} {H}_{i} + α {G}_{p} {H}_{P} - {δ}_{A} A ( {r}_{i} + {r}_{p} )$$

$$\frac{{dG}_{p}}{dt} = D ( {G}_{i} - {G}_{p} ) + {D}_{E} ( {G}_{E} - {G}_{p} ) - α {G}_{p} {H}_{p} - {δ}_{G} {G}_{p} {r}_{p}$$

$$\frac{{dH}_{i}}{dt} = {β}_{0} + {β}_{H} \frac{{G}_{i}^{n}}{{K}_{H}^{ n} + {G}_{i}^{ n}} - {γ}_{H} {H}_{i}$$

$$\frac{{dH}_{p}}{dt} = {β}_{0} - {γ}_{H} {H}_{p}$$

FIG. 4e shows the model prediction: in agreement with the experiments, overexpressing GDH leads to quenching of oscillation. A systematic analysis on different levels of overexpression is shown in the bifurcation diagram in FIG. 10e.

### Addition of Hydrogen Peroxide to the Media

Hydrogen peroxide is a strong oxidizer that can kill the cells on the periphery of the biofilm. Dead cells in the biofilm still affect glutamate diffusion, but are metabolically inactive. Thus, the killing is implemented in the model by removing the production term of housekeeping proteins in the peripheral cell population. Additionally, a new negative term in the cellular density equation is introduced to account for cell death. To that end, the differential equations for rₚ and ρₚ are modified as shown below:

$$\frac{{dr}_{p}}{dt} = - {γ}_{r} {r}_{p}$$

$$\frac{d {ρ6}_{p}}{dt} = η {r}_{p} {ρ}_{p} ( 1 - \frac{{ρ}_{p}}{K ( {G}_{p} )} ) - {γ}_{{H}_{2} {0}_{2}} {ρ}_{p} - {λ}_{p} {ρ}_{p}$$

The new term is also added to the equation for the rate population expansion:

$${μ}_{p} = η {r}_{p} {ρ}_{p} ( 1 - \frac{{ρ}_{p}}{K ( {G}_{p} )} ) - {λ}_{{H}_{2} {0}_{2}} {ρ}_{p}$$

Finally, in the case of GDH overexpression, hydrogen peroxide entirely eliminates GDH production in the peripheral cell population, and the differential equation for Hₚ becomes:

$$\frac{{dH}_{p}}{dt} = - {γ}_{H} {H}_{p}$$

FIG. 4h shows the model prediction on the average growth rate and death in interior and peripheral populations after the addition of hydrogen peroxide, for both wild type and GDH overexpressing biofilms.

### Effect of Varying the Ratio of Interior to Peripheral Cells

As a consequence of biofilm expansion the relative size of interior and peripheral cell populations changes over time. Since the variables of the mathematical model represent intensive quantities (their value does not depend on the total volume) most of the equations are not affected by changes in the relative size of both cell populations. The only exception is the equation for ammonium, as it describes the concentration of this species in the whole biofilm, taking into account reactions that occur exclusively in one or the other population region. In this case the relative size of each one of these two regions will modulate the relative effect of these reactions.

To explore the effects of changes in the relative sizes of the two populations, ƒᵢ is defined as the fraction of the size of the interior population over the whole biofilm population. This parameter allows us to distinguish the contributions of the interior and peripheral regions to both the production and the consumption of ammonium:

$$\frac{dA}{dt} = {f}_{i} α {G}_{i} {H}_{i} - {δ}_{A} A ( {f}_{i} {r}_{i} + ( 1 - {f}_{i} ) {r}_{p} )$$

This equation allows determining the effect of an increase in ƒᵢ (such as the one that occurs in the biofilm as it expands) on the growth oscillations reported above. FIG. 10f shows that the oscillations persist for a wide range of ƒᵢ values, with a period that increases only slightly with ƒᵢ, in agreement with the experimental observations.

### Sensitivity Analysis

FIGS. 10g and 10h show how changes in each one of the intrinsic parameters of the model affect the period and the modulation depth of the oscillations. The values of the parameters were scanned from 50% to 150% of its original value. Whenever a modulation depth lower than 2% was measured the system was considered to be non-oscillating, and labeled in gray in the color plot.

### Example 7

### Method of Treatment and/or Control of Biofilms in Oil Pipelines

Biofilms that form in pipelines can severely clog fluid flow. The invention treatment method used to eliminate biofilms formed in pipelines, comprising the following steps:

(1) Generating metabolic codependence within the biofilm: Flow a media comprised of minimal salts supplemented only with glycerol (0.5% weight/volume) and glutamate (0.5% weight/volume). This media triggers metabolic codependence of the growth of peripheral cells on ammonium produced by protected interior cells. This media is completely non-toxic to humans;

(2) Establishing a successful development of metabolic codependence within the biofilm community. To determine successful establishment of metabolic codependence, examine metabolite concentration contained in the fluid flow coming out of the pipeline. The signature of metabolic codependence is fluctuations in glutamate consumption (concentration) by the biofilm;

(3) Inducing an unrestricted growth of biofilm peripheral cells that lethally starves the protected interior cells. Expose the pipeline, and thereby the biofilms within, to either ammonia gas or ammonium solution for two to six hours (depending on degree of contamination). Supplementation with ammonium or ammonia makes growth of peripheral cells independent from metabolites provided by interior cells. As a result, persistent growth of peripheral cells consumes the nutrients before they reach interior cells, lethally starving them;

(4) Eliminating accessible peripheral cells. Once the protected interior cells are starved to death, the remaining peripheral cells can easily be eliminated by applying 1-4% hydrogen peroxide solution (also depending on level of contamination). Since interior cells are already dead, killing of peripheral cells after ammonium treatment minimizes damage to piping, while maximizing killing efficacy; and

(5) Verifying treatment effectiveness. After treatment is complete, samples of fluid flown through the pipeline will be used to assess the number of remaining viable bacteria within the pipeline.

### REFERENCES

[1] Ben-Jacob, E., Cohen, I., and Levine, H. (2000) Cooperative self-organization of microorganisms, Adv Phys 49, 395-554.

[2] Eldar, A. (2011) Social conflict drives the evolutionary divergence of quorum sensing, Proceedings of the National Academy of Sciences of the United States of America 108, 13635-13640.

[3] Gregor, T., Fujimoto, K., Masaki, N., and Sawai, S. (2010) The Onset of Collective Behavior in Social Amoebae, Science 328, 1021-1025.

[4] Wingreen, N. S., and Levin, S. A. (2006) Cooperation among microorganisms, Plos Biol 4, 1486-1488.

[5] Hibbing, M. E., Fuqua, C., Parsek, M. R., and Peterson, S. B. (2010) Bacterial competition: surviving and thriving in the microbial jungle, Nature reviews. Microbiology 8, 15-25.

[6] Oliveira, N. M., Niehus, R., and Foster, K. R. (2014) Evolutionary limits to cooperation in microbial communities, Proceedings of the National Academy of Sciences of the United States of America 111, 17941-17946.

[7] Davies, D. (2003) Understanding biofilm resistance to antibacterial agents, Nature reviews. Drug discovery 2, 114-122.

[8] Donlan, R. M., and Costerton, J. W. (2002) Biofilms: survival mechanisms of clinically relevant microorganisms, Clinical microbiology reviews 15, 167-193.

[9] Vlamakis, H., Aguilar, C., Losick, R., and Kolter, R. (2008) Control of cell fate by the formation of an architecturally complex bacterial community, Genes & development 22, 945-953.

[10] Yildiz, F. H., and Visick, K. L. (2009) Vibrio biofilms: so much the same yet so different, Trends in microbiology 17, 109-118.

[11] Berk, V., Fong, J. C., Dempsey, G. T., Develioglu, O. N., Zhuang, X., Liphardt, J., Yildiz, F. H., and Chu, S. (2012) Molecular architecture and assembly principles of Vibrio cholerae biofilms, Science 337, 236-239.

[12] Costerton, J. W., Stewart, P. S., and Greenberg, E. P. (1999) Bacterial biofilms: a common cause of persistent infections, Science 284, 1318-1322.

[13] Hall-Stoodley, L., Costerton, J. W., and Stoodley, P. (2004) Bacterial biofilms: from the natural environment to infectious diseases, Nature reviews. Microbiology 2, 95-108.

[14] Asally, M., Kittisopikul, M., Rue, P., Du, Y., Hu, Z., Cagatay, T., Robinson, A. B., Lu, H., Garcia-Ojalvo, J., and Suel, G. M. (2012) Localized cell death focuses mechanical forces during 3D patterning in a biofilm, Proceedings of the National Academy of Sciences of the United States of America 109, 18891-18896.

[15] Wilking, J. N., Zaburdaev, V., De Voider, M., Losick, R., Brenner, M. P., and Weitz, D. A. (2013) Liquid transport facilitated by channels in Bacillus subtilis biofilms, Proceedings of the National Academy of Sciences of the United States of America 110, 848-852.

[16] Branda, S. S., Gonzalez-Pastor, J. E., Ben-Yehuda, S., Losick, R., and Kolter, R. (2001) Fruiting body formation by Bacillus subtilis, Proceedings of the National Academy of Sciences of the United States of America 98, 11621-11626.

[17] Gunka, K., and Commichau, F. M. (2012) Control of glutamate homeostasis in Bacillus subtilis: a complex interplay between ammonium assimilation, glutamate biosynthesis and degradation, Molecular microbiology 85, 213-224.

[18] Stannek, L., Thiele, M. J., Ischebeck, T., Gunka, K., Hammer, E., Volker, U., and Commichau, F. M. (2015) Evidence for synergistic control of glutamate biosynthesis by glutamate dehydrogenases and glutamate in Bacillus subtilis, Environmental microbiology.

[19] Belitsky, B. R., and Sonenshein, A. L. (1998) Role and regulation of Bacillus subtilis glutamate dehydrogenase genes, Journal of bacteriology 180, 6298-6305.

[20] Zeigler, D. R., Pragai, Z., Rodriguez, S., Chevreux, B., Muffler, A., Albert, T., Bai, R., Wyss, M., and Perkins, J. B. (2008) The origins of 168, W23, and other Bacillus subtilis legacy strains, Journal of bacteriology 190, 6983-6995.

[21] Nakano, M. M., Yang, F., Hardin, P., and Zuber, P. (1995) Nitrogen regulation of nasA and the nasB operon, which encode genes required for nitrate assimilation in Bacillus subtilis, Journal of bacteriology 177, 573-579.

[22] Kleiner, D. (1985) Bacterial Ammonium Transport, Ferns Microbiol Lett 32, 87-100.

[23] Castorph, H., and Kleiner, D. (1984) Some properties of a Klebsiella pneumoniae ammonium transport negative mutant (Amt-), Archives of microbiology 139, 245-247.

[24] Boogerd, F. C., Ma, H. W., Bruggeman, F. J., van Heeswijk, W. C., Garcia-Contreras, R., Molenaar, D., Krab, K., and Westerhoff, H. V. (2011) AmtB-mediated NH3 transport in prokaryotes must be active and as a consequence regulation of transport by GlnK is mandatory to limit futile cycling of NH4+/NH3, Febs Lett 585, 23-28.

[25] Jayakumar, A., Schulman, I., MacNeil, D., and Barnes, E. M., Jr. (1986) Role of the Escherichia coli glnALG operon in regulation of ammonium transport, Journal of bacteriology 166, 281-284.

[26] Kim, M., Zhang, Z., Okano, H., Yan, D., Groisman, A., and Hwa, T. (2012) Need-based activation of ammonium uptake in Escherichia coli, Molecular systems biology 8, 616.

[27] Commichau, F. M., Gunka, K., Landmann, J J, and Stulke, J. (2008) Glutamate metabolism in Bacillus subtilis: gene expression and enzyme activities evolved to avoid futile cycles and to allow rapid responses to perturbations of the system, Journal of bacteriology 190, 3557-3564.

[28] Detsch, C., and Stulke, J. (2003) Ammonium utilization in Bacillus subtilis: transport and regulatory functions of NrgA and NrgB, Microbiology 149, 3289-3297.

[29] Anyan, M. E., Amiri, A., Harvey, C. W., Tierra, G., Morales-Soto, N., Driscoll, C. M., Alber, M. S., and Shrout, J. D. (2014) Type IV pili interactions promote intercellular association and moderate swarming of Pseudomonas aeruginosa, Proceedings of the National Academy of Sciences of the United States of America 111, 18013-18018.

[30] Irnov, I., and Winkler, W. C. (2010) A regulatory RNA required for antitermination of biofilm and capsular polysaccharide operons in Bacillales, Molecular microbiology 76, 559-575.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and methods disclosed herein are specifically described, other combinations of the compositions and methods also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A method of controlling the growth of a biofilm having periphery cells and interior cells, comprising:
a) generating metabolic codependence within the biofilm,
b) establishing development of metabolic codependence within the biofilm,
c) inducing unrestricted growth of the periphery cells in an amount sufficient to starve the interior cells to death, and
d) treating and eliminating accessible periphery cells, or a combination thereof.

2. The method of claim 1, wherein the growth of the periphery cells is promoted by supplying a media comprising salts supplemented with glycerol and glutamate.

3. The method of claim 2, wherein the glycerol is about 0.5% weight/volume.

4. The method of claim 2, wherein the glutamate is about 0.5% weight/volume.

5. The method of claim 2, wherein the media is non-toxic to humans.

6. The method of claim 1, wherein the growth of the periphery cells is promoted by manipulating the activity of metabolic enzyme glutamate dehydrogenase (GDH).

7. The method of claim 1, wherein the biofilm is exposed to ammonium, ammonia gas, or a combination thereof, in a sufficient amount and for a sufficient period of time, wherein the growth of peripheral cells is independent from nutrients provided by interior cells of the biofilms.

8. The method of claim 7, wherein the nutrient is ammonium.

9. The method of claim 1, wherein the accessible peripheral cells are treated and eliminated with a solution comprising an effective amount of a biofilm control substance.

10. The method of claim 9, wherein the biofilm control substance comprises an antibiotic or toxic chemical.

11. The method of claim 10, wherein the toxic chemical is hydrogen peroxide.

12. The method of claim 1, wherein the biofilm is a microbial colony comprising Bacillus subtilis.

13. A method of controlling or treating biofilm infections in clinical medicine comprising the method of claim 1.

14. A method of controlling or treating biofilm formed on teeth in dental medicine comprising the method of claim 1.

15. A method of treating biofilms colonized on medical equipment comprising the method of claim 1.

16. A method of controlling biofilm contamination during food processing and storage comprising the method of claim 1.

17. A method of treating or controlling biofilms clog in oil pipelines or a pipeline of a water supply system, comprising:
a) generating metabolic codependence of the growth of peripheral cells of the biofilms on ammonium produced by protected interior cells within the biofilms;
b) establishing successful development of metabolic codependence of fluctuations in glutamate consumption within the biofilm;
c) inducing unrestricted growth of biofilm peripheral cells that consume the nutrients for interior cells, causing the interior cells starved to death;
d) eliminating accessible peripheral cells; and
e) verifying treatment effectiveness.

18. The method of claim 17, wherein the accessible peripheral cells are eliminated by about 1-4% hydrogen peroxide solution.
